(19) United States
(12) Patent Application Publication
(10) Pub. No.: US 2011/0275837 A1
(43) Pub. Date: Nov. 10, 2011

(54) Waxes Having High Polarity and the Use Thereof as Slip Agents for Chlorine-Containing Thermoplastics

(75) Inventors: Gerd Hohner, Gersthofen (DE); Beate Treffler, Neusaess (DE)

(73) Assignee: CLARIANT FINANCE (BVI) LIMITED, Tortola (VG)

(21) Appl. No.: 13/144,787

(22) PCT Filed: Feb. 5, 2010
(86) PCT No.: PCT/EP10/00720

§ 371 Date: Jul. 15, 2011

(30) Foreign Application Priority Data
Feb. 10, 2009 (DE) 10 2009 008 257.3
Feb. 5, 2010 (EP) PCT/EP2010/000720

(51) Int. Cl. C07D 307/36

(52) U.S. Cl. 549/255

(57) ABSTRACT

Copolymer waxes, characterized by an iodine number of <15, preferably <13 g of iodine/100 g, a viscosity at 100° C. ranging between 200 to 4000 mPa*s, a dropping point ranging between 60 to 90° C., a ram penetration hardness ranging between 200 to 600 bar, an acid number ranging between 60 to 100 mg of KOH/g, and an iodine color number<10. The copolymer waxes according to the invention are suited in particular as processing aids for PVC, polyvinylidene chloride and copolymers of vinyl chloride, having up to 30% by weight comonomers.

The invention relates to high-polarity waxes produced via free-radical-initiated reaction of long-chain olefin hydrocarbons with unsaturated polycarboxylic acids or with their anhydrides, and also to the use of these waxes as processing aids for chlorine-containing thermoplastics.

Processing of thermoplastics usually uses lubricants, which firstly improve the rheology of the polymer melt and secondly are intended to reduce its tendency to stick to the metallic parts of the processing machines. Lubricants are particularly important in the processing of chlorine-containing thermoplastics, e.g. polyvinyl chloride (PVC), since these plastics are easily damaged by thermal stress and high shear forces and are very susceptible to adhesion, and cannot therefore be processed without lubricant.

A distinction is made between those lubricants which have good compatibility with the polymer melt and therefore act primarily to improve flow (internal lubricants) and those which have some degree of incompatibility and therefore become concentrated at the phase boundaries, where they exhibit a release effect (external lubricants).

There is a wide variety of available products with internal lubricant effect for improving the rheology of PVC melts, examples being fatty alcohols, fatty acids, full or partial esters of fatty acids, and amides of fatty acids.

A more difficult area is the selection of lubricants with external, or combined external and internal, lubricant effect, where these are sometimes subject to conflicting requirements. In order to be effective they have to have some degree of incompatibility with the PVC melt, so that a film providing effective release action can be produced between the melt and the metal parts of the processing machines. However, a disadvantage of incompatible additives is that they frequently cause severe haze in the final product. In the numerous applications where a highly transparent final product is desirable, the amount that can be used of a lubricant is therefore subject to a limit such that its effect is inadequate. This is particularly disadvantageous in the production of thin calendered PVC foils, these are susceptible to adhesion and have low thermal stability in the hot state, and on the one hand release from the rolls requires a large amount of lubricant, but on the other hand there are particularly stringent transparency requirements placed upon foils of this type, e.g. in the packaging sector.

Another precondition for the ability to use lubricant additives consists in sufficiently low volatility and high thermal stability under the conditions of processing. There must be no possibility that the additives can be converted to volatile substances at the usual temperatures used in calendering, extrusion, or injection molding, either because their vapor pressures are excessive, for example because of small molecular size, or because volatile decomposition products are produced as a consequence of inadequate thermochemical stability. Volatility can easily be determined, e.g. via thermogravimetric analysis (TGA). Another important factor for the quality of a lubricant additive is that its does not discolor the plastics composition. Experience has shown that even the intrinsic color of the additive has a considerable effect on the color of the final item.

Solid lubricants are usually used in the form of powders, which are produced via spraying or milling. To the extent that grinding processes are used, the products have to have a certain hardness in order that they can be ground in conventional industrial mills. There are methods available for characterizing hardness properties, for example measurement of plunger penetration.

Montan waxes are known lubricants for producing plasticizer-free PVC foils. Although these have a balanced effectiveness profile, their production is complicated. The starting material here is crude wax, which is obtained from wax-containing brown coal via solvent extraction, and the constitution of which can vary, because it comes from a natural source. Further processing, after additional extractive purification, uses expensive chromic-sulfuric acid treatment, and the result of profound chemical changes here is product mixtures composed mainly of long-chain carboxylic acids. These are in turn converted in subsequent synthesis steps to wax esters or wax soaps, which are used as “semisynthetic” lubricant waxes.

Other known PVC lubricants are those known as complex or mixed esters derived from aliphatic or aromatic dicarboxylic acids having from 2 to 22 carbon atoms, from aliphatic polyols having from 2 to 6 hydroxy groups, and from aliphatic monocarboxylic acids having from 12 to 30 carbon atoms (GB 1292548), and also mixed esters derived from aliphatic diols, from aliphatic or aromatic polycarboxylic acids having from 2 to 6 carboxy groups, and from aliphatic monofunctional alcohols having from 12 to 30 carbon atoms (GB 1450273).

EP 0 545 306 describes copolymers which are suitable as PVC lubricants and which derive from C₁₂-C₆₀alpha-olefins, from unsaturated monocarboxylic acids, from unsaturated monocarboxylic esters, and optionally styrene, therefore deriving from at least three different monomers.

There are also known PVC lubricants obtainable via copolymerization of relatively long-chain alpha-olefins and maleic anhydride. By way of example, DE-A 2 748 367 discloses copolymers of olefins having from 8 to 24 carbon atoms and maleic anhydride. Said products exhibit comparatively high volatility values and are soft; further processing via milling is therefore impossible.

DE-A 3 003 797 describes a forming process for plastics, specifically for PVC. The lubricant used comprises an esterification-derivatized copolymer of unsaturated polycarboxylic acids and, respectively, their anhydrides with alpha-olefins. One of the examples also uses, alongside the ester products of the invention, an underivatized copolymer of C₃₀+ olefin and maleic anhydride as lubricant in PVC. The copolymer is produced via reaction of the olefin with, based on C₃₀+ olefin used, 23.3% by weight of maleic anhydride and 1.9% by weight of di-tert-butyl peroxide. The product has a comparatively dark intrinsic color and when used in PVC gives molding compositions with high Yellowness Index.

Although the abovementioned materials have some degree of effectiveness as PVC lubricants, they exhibit specific disadvantages.

Reaction products derived from long-chain alpha-olefins with maleic anhydride are also described in DE-A 3 510 233.

EP 1 693 047 discloses copolymer waxes for cosmetic preparations; these are produced from C₂₆-C₆₀ alpha-olefins and maleic anhydride.

Surprisingly, it has now been found that copolymer waxes produced from long-chain alpha-olefins and from polycarboxylic acids or their anhydrides are particularly advantageously suitable as PVC lubricants if they are produced under certain conditions and have specific properties.

The present invention therefore provides copolymer waxes which have

an iodine number of <15 g of iodine/100 g, preferably <13 g of iodine/100 g,
a viscosity at 100° C. of from 200 to 4000 mPa*s,
a drop point from 60 to 90° C.,
a plunger penetration hardness from 200 to 600 bar,
an acid number from 60 to 100 mg of KOH/g, and also
an iodine color number<10.

The invention further provides a process for producing these copolymer waxes via reaction of alpha-olefins having chain lengths greater than or equal to 28 carbon atoms and of unsaturated polycarboxylic acids or their anhydrides in the presence of, based on olefin hydrocarbon used, at least 2.0% by weight of a free-radical initiator.

Alpha-olefins that can be used are those having chain lengths from 28 to 60 carbon atoms, preferably from 30 to 60 carbon atoms. It is possible to use either olefins having uniform chains or olefin mixtures, for example those arising as distillation cuts or distillation residues in the known production processes during the catalytic oligomerization of ethylene. Technical alpha-olefin mixtures, in particular those having relatively high chain length, can comprise not only 1-alkenes but varying amounts of internal and pendant olefinic double bonds (vinylene groups and vinylidene groups).

Representative examples of the unsaturated polycarboxylic acids and, respectively anhydrides used for the reaction with the alpha-olefins are maleic acid, fumaric acid, citraconic acid, mesaconic acid, aconitic acid, or itaconic acid and, respectively, the anhydrides of said polycarboxylic acids, to the extent that these are obtainable. Preference is given to maleic anhydride. It is also possible to use mixtures of said polycarboxylic acids and/or anhydrides in any desired ratios. The usage ratio of the polycarboxylic acids and, respectively, anhydrides to the alpha-olefin starting component is from 1:8 to 1:5 parts by weight, preferably from 1:7 to 1:5.5 parts by weight. The amount used of polycarboxylic acid and, respectively, anhydride, based on alpha-olefin, is accordingly from 12.5 to 20% by weight, preferably from 14.3 to 18.2% by weight.

The lubricants and release agents of the invention are produced in a manner known per se via reaction of the abovementioned components at elevated temperature with addition of organic or inorganic free-radical-forming initiators. The reaction can be carried out in the presence of, or in the absence of, a solvent. The latter method is preferred. The reaction can moreover be carried out either batchwise, e.g. in a stirred tank, or in a continuously operating reactor.

Examples of suitable organic initiators are peroxides, e.g. alkyl hydroperoxides, or dialkyl or diaryl peroxides, diaroyl peroxides, peresters, or azo compounds. Preference is given to dialkyl peroxides, and di-tert-butyl peroxide is specifically preferred. However, it is also possible to use any other initiator, as long as it decomposes into free radicals at the selected reaction temperature and is capable of initiating the reaction.

When the reaction is carried out in the absence of solvent, the reaction temperatures are above the melting point of the alpha-olefin, e.g. from 100 to 200° C., preferably from 120 to 180° C., particularly preferably from 140 to 170° C. The copolymer waxes of the invention are used in chlorine-containing thermoplastics, e.g. polyvinyl chloride, as lubricants or release agents; the thermoplastics can be produced by known processes—examples being suspension polymerization, bulk polymerization, and emulsion polymerization. They are suitable not only for polyvinyl chloride but also for copolymers of vinyl chloride with up to 30% by weight of comonomers, examples being vinyl acetate, vinylidene chloride, vinyl ether, acrylonitrile, esters of acrylic acid, mono- or diesters of maleic acid, or olefins, and also for graft copolymers of PVC.

The amount added is from 0.05 to 5.0% by weight, based on the polymer. If the molding composition is based on MPVC or SPVC (bulk PVC or suspension PVC), the amount added is preferably from 0.05 to 1% by weight, but if it is based on EPVC (emulsion PVC), said amount is preferably from 1.0 to 5% by weight, in particular from 2 to 4% by weight, based in each case on the polymer. Mixing to incorporate the copolymers of the invention into the polymers is achieved in the usual manner during the production or processing of the molding compositions.

The PVC composition can also comprise, alongside the copolymer waxes of the invention, fillers, heat stabilizers, antioxidants, light stabilizers, antistatic agents, flame retardants, reinforcing materials, pigments, dyes, processing aids, lubricants, impact modifiers, blowing agents, or optical brighteners, in conventional amounts.

### Inventive Examples

Melt viscosities were determined to DIN 53019-2 with a rotary viscometer, and drop points were determined to DIN ISO 2176. Acid number was determined to DIN 53402, except that the solvents used were anhydrous ethanol and xylene, with the aim of avoiding hydrolytic cleavage of the anhydride groups. Saponification number was measured in accordance with DIN standard 53401, iodine number was determined in accordance with DIN standard 14111, and iodine color number was determined to DIN 6162. TGA measurements were carried out with a Mettler SD TA 551e system (temperature program: heating from room temperature to 300° C., heating rate 5°/min, nitrogen flow rate 50 ml/min). Plunger penetration, as a measure of hardness, was measured by DGF method M-III 9e (see also Fiebig, Braun, Fett/Lipid 98, 1996, No. 2, 86).

### Production of Copolymer Waxes

2500 g of C₃₀+ alpha-olefin (mixture of olefin hydrocarbons having chain lengths in essence of >=30 carbon atoms; commercially available product from ChevronPhillips) are melted under nitrogen in a glass apparatus equipped with stirrer unit, internal thermometer, and distillation system. The amount stated in table 1 of maleic anhydride was then added—divided into six identical portions, at intervals of in each case 30 min. Within the same period, di-tert-butyl peroxide was added continuously from a dropping funnel. The reaction was then allowed to continue for 1 h. Volatile fractions were then removed by distillation in vacuo (about 30 mbar). After about 30 min, nitrogen was introduced to bring the system to atmospheric pressure. Table 1 lists the data for the resultant copolymer waxes.

### Comparative Examples

Copolymers known from the literature, derived from C₃₀+ olefin and maleic anhydride, were produced in accordance with the instructions given in DE-A 3 003 797, example 1, DE-A 3 510 233, example 1, and also EPA 1 693 047, ex. A (copolymer wax 1). Table 2 lists the measured data.

TABLE 1
Copolymer waxes derived from C₃₀+ olefin and maleic anhydride

| | Amount of | Amount of di- | | | | | | | | |
|---|---|---|---|---|---|---|---|---|---|---|
| | maleic | tert-butyl | | | Vis- | | Iodine | | Plunger | Loss of mass |
| | anydride | peroxide | Reaction | Acid | cosity, | Iodine | color | Drop | pene- | at 260° C. in |
| Example | used¹⁾ | used¹⁾ | temp. | number | 100° C. | number | number | point | tration | TGA |
| 1 | 15.0 | 2.0 | 160 | 80 | 329 | 8.0 | 3.9 | 74 | 410 | 2.0 |
| 2 | 15.0 | 3.0 | 160 | 83 | 608 | 7.1 | 2.9 | 72 | 540 | 2.6 |
| 3 | 18.0 | 3.0 | 160 | 93 | 2767 | 6 | 3.7 | 73 | 535 | 2.7 |
| comp 1 | 15.0 | 1.0 | 160 | 78 | 260 | 16 | 2.7 | 73 | 337 | 3.7 |
| (not | | | | | | | | | | |
| inventive) | | | | | | | | | | |
| | % by wt. | % by wt. | ° C. | mg KOH/g | mPa*s | g I2/100 g | | ° C. | bar | % by wt. |

¹⁾based on C₃₀+ olefin used

TABLE 2
Copolymer waxes derived from C₃₀+ olefin and maleic anhydride corresponding to the prior art

| | Amount of | Amount of di- | | | | | | | |
|---|---|---|---|---|---|---|---|---|---|
| | maleic | tert-butyl | | | Vis- | | Iodine | | Loss of mass |
| Comparative | anhydride | peroxide | Reaction | Acid | cosity | Iodine | color | Drop | at 260° C. in |
| example | used¹⁾ | used¹⁾ | temp. | number | 100° C. | number | number | point | TGA |
| comp 2 | 15.0 | 0.4 | 180 | 77 | 162 | 18 | 3.8 | 73 | 4.9 |
| (corresponding to | | | | | | | | | |
| DE3510233, ex. 1) | | | | | | | | | |
| comp 3 | 23.3 | 1.9 | 200 | 111 | 1498 | 5.2 | 12 | 74 | 2.1 |
| (corresponding to | | | | | | | | | |
| DE3003797, ex. 1) | | | | | | | | | |
| comp. 4 | 18.4 | 0.92 | 160 | 92 | 428 | 9 | 9.2 | 74 | 4.4 |
| (corresponding to | | | | | | | | | |
| EP1693047 | | | | | | | | | |
| ex. A/copolymer wax | | | | | | | | | |
| 1) | | | | | | | | | |
| | % by wt. | % by wt. | ° C. | mg KOH/g | mPa*s | g I2/100 g | | ° C. | % by wt. |

¹⁾based on C₃₀+ olefin used

### Comparative Example Comp 5

A copolymer wax was produced by analogy with EP 0 545 306, example 1, from C₃₀+ olefin, methyl acrylate, and acrylic acid. Unlike in the example described, a C₃₀+ olefin was used instead of C₂₄-C₆₀ olefin. There were no changes to the other components or to the quantitative proportions, reaction conditions, and procedures.

The properties of the copolymer wax were as follows:

Acid number: 12 mg KOH/g
Saponification number: 163 mg KOH/g
Drop point: 73° C.
Viscosity at 90° C.: 610 mPa*s

The following commercially available PVC lubricants were used for the performance tests (comparative examples):

| | | Loss of mass at 260° C. |
|---|---|---|
| | Producer | in TGA |
| Licowax E | Clariant | 4.0 |
| Loxiol G 74 | Cognis | 14.6 |
| | | % by wt. |

### Performance Tests

PVC formulations corresponding to the constitution stated in table 3 were premixed in a laboratory mixer and then processed to give milled sheets on a Collin 150 M data-gathering roll mill (Dr. Collin GmbH). 220 g of the respective PVC mixture comprising additives were used in each case here, and the processing temperature was 195° C. After 20 min of running time, adhesion and Yellowness Index (YI) were measured. The mixtures of the respective test formulations were moreover pressed to give sheets of thickness 2 mm (Collin 200P laboratory sheet press, press temperature 180° C., press pressure 200 bar). The transparency of said sheets was measured.

TABLE 3
Test PVC formulation

| | |
|---|---|
| PVC S 3160 (K value 60, Vinnolit) | 100 |
| Kane Ace B 58 (Impact modifier, Kaneka) | 5 |
| Mark 17 MOK (Tin stabilizer, Crompton) | 1.5 |
| Paraloid K 120N (Processing aid, Rohm and Haas) | 1 |
| Loxiol G 16 (Glycerol dioleate, Cognis) | 0.5 |
| Lubricant as in tables 1-3 | 0.3 |
| | phr |

TABLE 4
Results of performance tests

| | Yellowness Index | Adhesion after | |
|---|---|---|---|
| | after 20 min | 20 min | Transparency |
| Copolymer wax | 28 | 3.9 | 80 |
| from example 1 | | | |
| Copolymer wax | 23 | 4.7 | 79 |
| from example 3 | | | |
| Licowax E | 35 | 6.2 | 83 |
| Loxiol G 74 | adheres | adheres | 83 |
| Comp 1 | 30 | 6.3 | 77 |
| Comp 3 | 43 | 4.6 | 73 |
| Comp 5 | adheres | adheres | 80 |

1. A copolymer wax comprising
an iodine number of <15 g of iodine/100 g,
a viscosity at 100° C. of from 200 to 4000 mPa*s,
a drop point from 60 to 90° C.,
a plunger penetration hardness from 200 to 600 bar,
an acid number from 60 to 100 mg of KOH/g, and
an iodine color number<10.

2. The copolymer wax as claimed in claim 1, produced via reaction of alpha-olefins having chain lengths greater than or equal to 28 carbon atoms and of unsaturated polycarboxylic acids or their anhydrides.

3. The copolymer wax as claimed in claim 2, wherein the chain length of the alpha-olefins is from 28 to 60 carbon atoms.

4. A process for producing a copolymer wax as claimed in claim 1 comprising the steps of reacting alpha-olefins having chain lengths greater than or equal to 28 carbon atoms and one or more unsaturated polycarboxylic acids or their anhydrides in the presence of at least 2.0% by weight of a free-radical initiator, based on olefin hydrocarbon used.

5. The process for producing a copolymer wax, as claimed in claim 4, wherein the alpha-olefins used have been obtained via oligomerization of ethylene.

6. The process for producing a copolymer wax, as claimed in claim 4, wherein the one or more polycarboxylic anhydride used comprises maleic anhydride.

7. The process for producing a copolymer wax, as claimed in claim 4, wherein the reaction is carried out at reaction temperatures from 100 to 200° C.

8. The process for producing a copolymer wax, as claimed in claim 7, wherein the alpha-olefins and maleic anhydride are reacted with one another in a ratio by weight of from 5:1 to 8:1.

9. A composition comprising a copolymer wax as claimed in claim 1, wherein the composition is in the form of a processing aids for PVC, polyvinylidene chloride, or copolymers of vinyl chloride with up to 30% by weight of one or more comonomers, wherein the one or more comonomers are selected from the group of vinyl acetate, vinylidene chloride, vinyl ether, acrylonitrile, esters of acrylic acid, mono- or diesters of maleic acid, and The present invention therefore provides copolymer waxes which have
an iodine number of <15 g of iodine/100 g, preferably <13 g of iodine/100 g,
a viscosity at 100° C. of from 200 to 4000 mPa*s,
a drop point from 60 to 90° C.,
a plunger penetration hardness from 200 to 600 bar,
an acid number from 60 to 100 mg of KOH/g, and also
an iodine color number<10.
_olefins.

10. The copolymer wax as claimed in claim 1, wherein the iodine number is <13 g of iodine/100 g.

11. The process for producing a copolymer wax, as claimed in claim 4, wherein the reaction is carried out at reaction temperatures from 120 to 180° C.

12. The process for producing a copolymer wax, as claimed in claim 4, wherein the reaction is carried out at reaction temperatures from 140 to 170° C.
